(19) United States
(12) Patent Application Publication
(10) Pub. No.: US 2022/0128555 A1
(43) Pub. Date: Apr. 28, 2022

(54) APPARATUSES FOR PERFORMING RAPID DIAGNOSTIC TESTS

(71) Applicant: Detect, Inc., Guilford, CT (US)

(72) Inventor: Todd Roswech, Ivoryton, CT (US)

(73) Assignee: Detect, Inc., Guilford, CT (US)

(21) Appl. No.: 17/511,185

(22) Filed: Oct. 26, 2021

Related U.S. Application Data
(60) Provisional application No. 63/106,312, filed on Oct. 27, 2020.

(51) Int. Cl. G01N 33/543; B01L 3/00; G01N 33/569

(52) U.S. Cl. CPC G01N 33/54388; B01L 3/5023; B01L 3/523; G01N 2333/165; B01L 2400/0683; B01L 2300/0672; G01N 33/56983

(57) ABSTRACT

Diagnostic devices for performing diagnostic tests are provided, as well as methods that utilize the diagnostic devices, methods for manufacturing the diagnostic devices, and test kits for performing the diagnostic tests. The diagnostic devices include a vial, a rupturable container disposed in an internal cavity of the vial and containing a fluid, and a test and readout device in fluid communication with the internal cavity of the vial. The rupturable container is configured to rupture during a test procedure of the diagnostic test to enable the fluid to flow into the internal cavity of the vial. The rupturable container may include a rupturable ampoule or a frangible seal. The rupturable container may be ruptured by deformation of the vial or by piercing caused by a sharp object in the vial or a sample swab inserted in the vial.

### CROSS-REFERENCE TO RELATED APPLICATIONS

The present application claims the benefit of priority of U.S. Provisional Application No. 63/106,312 filed Oct. 27, 2020, entitled “APPARATUSES FOR PERFORMING RAPID DIAGNOSTIC TESTS” (Attorney Docket No. H0966.70049US00), the entire contents of which is incorporated by reference herein.

### FIELD

The technology of the present invention relates generally to test apparatuses, test kits, and methods of using the test apparatuses and/or the test kits to perform rapid diagnostic tests to detect the presence of one or more target nucleic-acid sequences.

### BACKGROUND

The ability to rapidly diagnose diseases—particularly highly communicable infectious diseases—is critical to preserving human health through early detection and containment of the infectious diseases until reliable preventive measures (e.g., vaccines) and/or medicinal treatments or cures are developed. Rapid testing is critical to determining infected individuals quickly and minimizing their interactions with others, in order to minimize the spread of the diseases. As one example, the high level of contagiousness, the high mortality rate, and the lack of an early treatment or vaccine for the coronavirus disease 2019 (COVID-19) have resulted in a pandemic that has already infected millions and killed hundreds of thousands of people. The existence of rapid, accurate diagnostic tests, useable for detecting COVID-19 as well as other diseases, could allow individuals infected with a disease to be quickly identified and isolated, which could assist with containment of the disease. In the absence of such diagnostic tests, diseases such as COVID-19 may spread unchecked throughout communities.

### SUMMARY

Provided herein are apparatuses and techniques for performing diagnostic tests useful for detecting one or more pathogens by detecting one or more target nucleic-acid sequences corresponding to the pathogens. The apparatuses and techniques described herein may be performed in a point-of-care (POC) setting or home setting by a lay person without specialized equipment and without training in laboratory procedures.

According to an aspect of the present technology, a diagnostic device for performing a rapid diagnostic test is provided. The diagnostic device may be comprised of: a vial; a first rupturable container disposed in an internal cavity of the vial and containing a first fluid; and a test and readout device in fluid communication with the internal cavity of the vial. The first rupturable container may be configured to rupture during a test procedure of the diagnostic test to enable the first fluid to flow into the internal cavity of the vial.

In some embodiments of this aspect, the diagnostic device may further be comprised of a rupturing device disposed in the internal cavity of the vial. The first rupturable container may be comprised of a rupturable ampoule containing the first fluid. The rupturable ampoule may be configured to rupture when the rupturing device is pushed against a surface of the rupturable ampoule at a force exceeding a rupture force.

In some embodiments of this aspect, the diagnostic device may further be comprised of a rupturable sample container configured to fit in the internal cavity of the vial. The internal cavity of the vial is configured such that, when the sample container is fully inserted in the internal cavity of the vial, the rupturing device ruptures the rupturable ampoule to allow the first fluid to flow into the internal cavity of the vial, and the rupturing device ruptures the rupturable sample container to allow a sample fluid to flow into the internal cavity of the vial. The first fluid may be a diluent fluid and may interact with the sample fluid interact in the internal cavity of the vial to form a diluted sample fluid. In some embodiments, the diagnostic device may further be comprised of: a cap configured to seal an opening of the internal cavity of the vial; and a conduit configured to enable the fluid communication between the internal cavity of the vial and the test and readout device. A location of the conduit may prevent the diluted sample fluid from reaching the conduit when the vial is in an upright position. In an inverted position with the cap sealing the opening of the internal cavity of the vial, the diluted sample fluid may be able to reach the conduit.

In some embodiments of this aspect, the diagnostic device may further be comprised of a lateral-flow assay (LFA) strip disposed in the test and readout device. An intake end of the LFA strip may be located proximate the conduit. The test and readout device may be comprised of a window through which the LFA strip is visible. In some embodiments, the diagnostic device may further be comprised of a sample pad located at the conduit and configured to delay movement of the diluted sample fluid to the LFA strip when the vial is in the inverted position.

In some embodiments of this aspect, the first rupturable container may be comprised of a first frangible seal confining the first fluid in a first portion of the internal cavity of the vial. In some embodiments, the diagnostic device may further be comprised of a reagent confined in a second portion of the internal cavity of the vial by a second frangible seal; a cap configured to seal an opening of the internal cavity of the vial; and a sample swab extending from an inner surface of the cap and sized to fit in the internal cavity of the vial with the cap sealing the end of the internal cavity of the vial. The sample swab may be configured to rupture the first and second frangible seals to fit in the internal cavity of the vial. In some embodiments, the reagent may be a lyophilized reagent or a liquid reagent. When the sample swab bearing a sample is inserted in the internal cavity of the vial with the cap sealing the opening of the internal cavity of the vial, the first fluid may be able to interact with the reagent and the sample to form a sample fluid.

In some embodiments of this aspect, the diagnostic device may further be comprised of: a housing; a crush effector disposed in the housing and configured to deform the vial to rupture the first rupturable container; and an actuator disposed in the housing and configured to control movement of the crush effector. The vial may be disposed in the housing and may be formed of a deformable material that enables a force to be transferred from the crush effector to the first rupturable container.

According to another aspect of the present technology, a diagnostic device for performing a rapid diagnostic test is provided. The diagnostic device may be comprised of: a vial configured to receive a sample container via an inlet opening of the vial; a rupturable ampoule disposed in the vial, the rupturable ampoule containing a liquid; a sharp object arranged in the vial and configured to pierce the vial and the rupturable ampoule; a test and readout device in fluid communication with the vial; a lateral-flow assay (LFA) strip disposed in the test and readout device; and a guide configured to convey a sample fluid from the vial to the test and readout device. In some embodiments, the guide may be comprised of a sample pad configured to guide movement of the sample fluid to the LFA strip.

According to a further aspect of the present technology, a diagnostic device for performing a rapid diagnostic test is provided. The diagnostic device may be comprised of: a housing; a deformable vial disposed in the housing; a first rupturable container disposed in an internal cavity of the vial and containing a first fluid; a second rupturable container disposed in the internal cavity of the vial and containing a second fluid; a test and readout device in fluid communication with the internal cavity of the vial; a lateral-flow assay (LFA) strip disposed in the test and readout device; and a crusher configured to deform the vial to rupture, individually or in unison, the first rupturable container and the second rupturable container. In some embodiments, the diagnostic apparatus may further be comprised of a guide configured to convey fluid from the vial to the test and readout device.

According to another aspect of the present technology, a diagnostic test method is provided. The method may be comprised of: rupturing a first rupturable container in an internal cavity of a vial of a diagnostic device, to enable a first fluid to flow into the internal cavity of the vial; enabling the first fluid to interact with a sample fluid to be tested, to form a sample solution; and permitting the sample solution to flow from the internal cavity of the vial to a conduit connecting the internal cavity of the vial with a testing compartment of the diagnostic device. In some embodiments, the permitting of the sample solution to flow from the internal cavity of the vial to the conduit may be comprised of: ensuring that an opening of the internal cavity at a first end of the vial is sealed, and inverting the vial to enable the sample solution to flow toward the first end of the vial. In some embodiments, a lateral-flow assay (LFA) strip may be disposed in the testing compartment such that an intake end of the LFA strip is proximate the conduit.

According to yet another aspect of the present technology, a method is provided for manufacturing a diagnostic device for performing a rapid diagnostic test. The method may be comprised of: obtaining a housing comprised of a vial and a testing compartment in fluid communication with the vial via a conduit; adding a first fluid to an internal cavity of the vial, the first fluid being contained in a first rupturable container; and enclosing a lateral-flow assay (LFA) strip in the testing compartment. In some embodiments, the method may further be comprised of: adding a second fluid to the internal cavity of the vial; and sealing an opening of the internal cavity of the vial with a removeable cap. In some embodiments, the first rupturable container may be a rupturable ampoule or a frangible seal. In some embodiments, the method may further be comprised of: positioning a sample pad at the conduit, wherein the sample pad is located in the vial or in the testing compartment or at least partially within the conduit.

According to further aspect of the present technology, a test kit for performing a rapid diagnostic test is provided. The test kit may be comprised of: a sample collection device; a diagnostic device comprised of: a vial, a first rupturable container disposed in an internal cavity of the vial and containing a first fluid, and a test and readout device in fluid communication with the internal cavity of the vial; and instructions for performing the rapid diagnostic test. The first rupturable container may be configured to rupture during a test procedure of the diagnostic test to enable the first fluid to flow into the internal cavity of the vial. In some embodiments, the test kit may further be comprised of: a rupturable sample container comprised of a second fluid. The sample container may be sized to fit in the vial, and the sample collection device may be configured to deliver a sample to the second fluid via an opening in the sample container. In some embodiments, the test kit may further be comprised of a reagent held in a caged cap. The caged cap may be configured to release the reagent to the sample container or to the vial when the caged cap is attached to the sample container or to the vial.

### BRIEF DESCRIPTION OF THE DRAWINGS

A skilled artisan will understand that the accompanying drawings are for illustration purposes only. It is to be understood that in some instances various aspects of the present technology may be shown exaggerated or enlarged to facilitate an understanding of the invention. In the drawings, like reference characters generally refer to like features, which may be functionally similar and/or structurally similar elements, throughout the various figures. The drawings are not necessarily to scale, as emphasis is instead placed on illustrating and teaching principles of the various aspects of the present technology. The drawings are not intended to limit the scope of the present teachings in any way.

FIG. 1A shows a perspective view of diagnostic device, according to some embodiments of the present technology.

FIG. 1B shows a perspective view of the diagnostic device of FIG. 1A in a partially disassembled state, according to some embodiments of the present technology.

FIG. 1C shows an elevational view of a cross section of the diagnostic device of FIG. 1A, according to some embodiments of the present technology.

FIG. 2A shows a perspective view of a swab device, according to some embodiments of the present technology.

FIG. 2B illustrates how a swab device may be used with a diagnostic device, according to some embodiments of the present technology.

FIG. 3A shows an elevational side view of a caged cap in a holding position, according to some embodiments of the present technology.

FIG. 3B shows a perspective view of the caged cap of FIG. 3A in a released position, according to some embodiments of the present technology.

FIG. 4 shows an elevational side view of a rupturable ampoule, according to some embodiments of the present technology.

FIG. 5 shows an elevational view of a cross section of a diagnostic device, according to some embodiments of the present technology.

FIGS. 6A through 6D show a sequence of acts that may be performed to load the diagnostic device of FIG. 5 with a sample contained in a rupturable device, according to some embodiments of the present technology.

FIG. 7 shows an elevational view of a cross section of a diagnostic device, according to some embodiments of the present technology.

FIG. 8A shows an elevational side view of a cross section of a rupturable vial, according to some embodiments of the present technology.

FIG. 8B shows an elevational side view of a cross section of a rupturable vial, according to some embodiments of the present technology.

FIG. 9 shows an elevational view of a cross section of a diagnostic device, according to some embodiments of the present technology.

FIG. 10 shows an elevational view of a cross section of a diagnostic device, according to some embodiments of the present technology.

### DETAILED DESCRIPTION

### 1. Introduction

The present disclosure provides test apparatuses, test kits, and methods of using the test apparatuses and/or the test kits (collectively referred to as “diagnostic systems” herein) for performing, in a clinical environment (e.g., medical facility, laboratory, etc.) and/or in a non-clinical environment (e.g., a home, a business office, a school, etc.), rapid diagnostic testing to detect one or more target nucleic-acid sequences. The diagnostic systems described herein, according to some embodiments of the present technology, may be self-administrable and may be comprised of any combination of: a sample-collecting device (e.g., a swab), reagents, a diagnostic device that enables a reaction between the reagents and a sample, and a detection component, which may be included as part of the diagnostic device.

According to some embodiments of the present technology, the sample-collecting device may be a disposable swab configured to contact a test subject to collect the sample and to transfer the collected sample to the diagnostic device, and then may be discarded. In some other embodiments, the sample-collecting device may comprise part of the diagnostic device and may participate in a procedure of the test. For example, the sample-collecting component may facilitate an interaction between the sample and one or more of the reagents.

According to some embodiments of the present technology, the detection component may be an assay vehicle (e.g., a strip) on which is contained or attached one or more reagents for detecting the presence of a target nucleic-acid sequence indicative of a particular pathogen or disease. In some embodiments, the assay vehicle may contain or have attached thereto a plurality of reagents for detecting the presence of a plurality of different target nucleic-acid sequences indicative of a plurality of different pathogens or diseases. In some embodiments, the assay vehicle may be a lateral-flow assay (LFA) strip configured to come into contact with a sample solution and to enable the sample solution to flow through the strip from one end to another. Observable changes in a region of the LFA strip may indicate the presence of the target nucleic-acid sequence, indicating that the test subject may be afflicted with the pathogen or disease corresponding to the target nucleic-acid sequence. In some instances, for LFA strips that are able to detect more than one pathogen or disease, observable changes in multiple regions of the LFA strip may indicate the presence of multiple target nucleic-acid sequences, indicating that the test subject may be afflicted with more than one pathogen or disease corresponding to the target nucleic-acid sequences. In some embodiments, the detection component may be incorporated in the diagnostic device to, for example, minimize handling by a user, who may be a person without medical training. For example, the diagnostic device may be comprised of a window that may enable changes in an assay vehicle to be visible, which may enable a user to perform a reading of a test result and/or may enable an image (e.g., a photograph) of the assay vehicle to be captured and automatically read by a computer algorithm.

According to some embodiments of the present technology, the reagents may be comprised of any one or any combination of one or more lysis reagents, one or more nucleic-acid amplification reagents, one or more CRISPR/Cas detection reagents. The reagents may be in solid form (e.g., lyophilized, crystallized, etc.) and therefore, in some embodiments, included with the reagents may be one or more buffer solutions configured to activate one or more of the reagents. Additionally, included with the reagents may be one or more diluent fluids for achieving a desirable concentration of reagent fluids during various procedures of the test.

According to some embodiments of the present technology, the diagnostic device may comprise components for handling the reagents prior to their use in the test, components for storing and/or handling the reagents or the sample, or mixtures thereof, during various procedures of the test, and components for promoting reactions between the sample and one or more of the reagents. For example, such components may include one or more reaction vessels (e.g., tubes, ampoules, vials, etc.) holding one or more reagents and/or one or more reaction fluids. In some embodiments, such components may be configured to be deformable (e.g., rupturable, crushable, burstable, etc.) to enable the one or more reagents and/or the one or more reaction fluids to mix with each other and/or with the sample during various procedures of the test. In some embodiments, such components may be physically part of or attached to the diagnostic device, and the diagnostic device may be referred to as a rupturable diagnostic device.

### 2. “Rupture-Type” Test Systems and Components

2.1 Vial Device with Frangible Seal

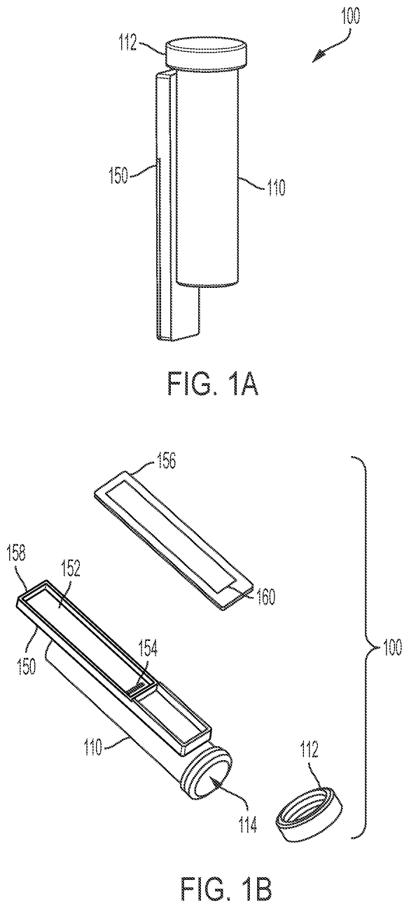

FIG. 1A depicts a perspective view of diagnostic device comprised of a vial device 100, according to some embodiments of the present technology. FIG. 1B depicts the vial device 100 in a partially disassembled state, according to some embodiments. FIG. 1C depicts an elevational view of a cross section of the vial device 100, according to some embodiments. The vial device 100 may be a rupturable diagnostic device because a rupturing procedure may occur during ordinary use of the vial device 100, as discussed below. According to some embodiments, the vial device 100 may be comprised of a reaction vial 110 and a test and readout device 150. The reaction vial 110 may be comprised of an internal cavity 116 in which a sample may interact with one or more reagents. During storage, an opening 114 of the internal cavity 116 may be covered by a removable cap 112.

According to some embodiments of the present technology, the reaction vial 110 may be comprised of one or more reagents located in the internal cavity 116. For example, a liquid 118 (e.g., a buffer solution, a reagent solution, a diluent liquid, etc.) may be confined to a portion of the internal cavity 116 by an interface 120. The interface 120 may prevent the liquid 118 from spilling out of the internal cavity 116. In some embodiments, the interface 120 may be a frangible seal 120 configured to keep the liquid 118 in the internal cavity 116 until the frangible seal 120 is ruptured or broken (e.g., pierced, torn, etc.) during a test procedure.

According to some embodiments of the present technology, the reaction vial 110 may be comprised of a solid r reagent 122 disposed in the internal cavity 116. The reagent 122 may be separated from the liquid 118 by the frangible seal 120. In some embodiments, the reagent 122 may be adhered to a surface of the frangible seal 120 and/or may optionally be confined to a compartment of the internal cavity 116 by a second interface 124, which may be a second frangible seal 124. For example, an inert gummy substance may be used to adhere the reagent 122 to an upper surface of the frangible seal 120, as shown in FIG. 1C, or to a lower surface of the second frangible seal 124). As will be appreciated, although FIG. 1C shows the liquid 118 to be located farther from the opening 114 of the reaction vial 110 than the reagent 122, in some embodiments the reagent 122 may be located farther from the opening 114 than the liquid 118, with the liquid 118 being confined, for example, between the first and second frangible seals 120, 124. In some embodiments, one or both of the first and second frangible seals 122, 124 may be comprised of a layer of metal foil, or a layer of a polymeric material, or a multilayer structure that includes a layer of metal foil and a layer of a polymeric material. In some embodiments, the reagent 122 may be a lyophilized reagent, and the liquid 118 may be a buffer solution configured to activate the reagent 122 during a test procedure. For example, during the test procedure the first and second frangible seals 120, 124 may be ruptured to enable the reagent 122 and the liquid 118 to interact with each other.

According to some embodiments of the present technology, the test and readout device 150 may be comprised of a testing chamber 152 configured to hold a LFA strip 180 (see FIG. 1C). The testing chamber 152 may include a conduit 154 configured to connect the testing chamber 152 with the internal cavity 116 of the reaction vial 110. In some embodiments, the conduit 154 may be configured to guide fluid from the internal cavity 116 toward the LFA strip 180. Although the conduit 126 is shown to be located at a central portion of the internal cavity 116 in FIGS. 1B and 1C), in some embodiments the conduit 126 may be located closer to a top end or a bottom end of the internal cavity 116. During assembly of the vial device 100, the LFA strip 180 may be sealed in the testing chamber 152 by a leak-tight cover 156, to prevent contamination of the LFA strip 180 and to prevent leakage of fluid during a test procedure. For example, the cover 156 may be fused to a base portion 158 of the test and readout compartment 150 by, e.g., welding, ultrasonic bonding, epoxy, and/or other techniques for providing a fluid-tight seal. In some embodiments, the cover 156 may be transparent or may have a transparent window 160 through which the LFA strip 180 may be visible to enable reading by a user or to enable an image to be captured for reading by an algorithm. For example, the LFA strip 180 may be positioned in the testing chamber 152 so that reaction stripes on the LFA strip 180 may be visible through the window 160 and read by an electronic reader (e.g., a camera operatively connected to a processor programmed to process image data obtained by the camera).

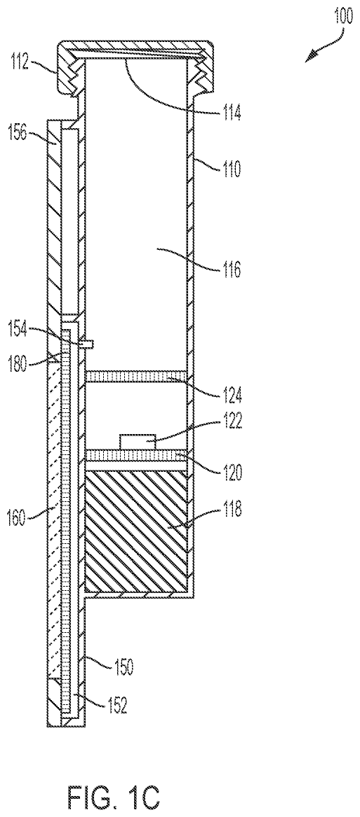

According to some embodiments of the present technology, a swab device 140 may be used with the vial device 100. As shown FIG. 2A, the swab device 140 may be comprised of a swab element 142 attached to a base 144. In some embodiments, the swab element 142 may be attached to the base 144 via a swab stem 146 extending from the base 144. A length of the swab stem 146 in a Z-direction and a cross-sectional dimension of the swab base 144 at or near a surface from which the swab stem 146 extends may be sized to prevent a user from, e.g., inserting the swab element 142 into a test subject's nasal cavity beyond a safe distance. The base 144 may have a length in the Z-direction suitable for reaching a desired distance into the internal cavity 116 of the reaction vial 110.

Optionally, according to some embodiments of the present technology, a swab cover 148 may be used to protect the swab element 142 during storage and/or transport, to maintain sterility of the swab element 142. In some embodiments, the swab cover 148 may be configured with protrusions (not shown) arranged to be received in corresponding indents (not shown) on the swab base 144 to provide a snap-fit structure, to cover both the swab element 142 and the swab stem 146 during transport and/or storage. The snap-fit structure may allow the swab cover 148 to be removed easily by a user when the user is ready to use the swab device 140 to collect a sample. As will be appreciated, other structures for holding the swab cover 148 onto the swab base 144 may be employed (e.g., a friction-fit structure, a screw-fit structure, etc.) instead of a snap-fit structure.

According to some embodiments of the present technology, the swab device 140 may have a swab cap 130, as shown in FIG. 2B. The swab cap 130 may be attached to an end of the swab base 144 opposite to an end on which the swab element 142 is located. In some embodiments, the swab cap 130 may serve as a handle for a user to hold the swab device 140 without touching the swab base 144. In some embodiments, the swab cap 130 may be configured to attached to a diagnostic device, to convey a sample collected on the swab element 142 into the diagnostic device. In some embodiments, the swab cap 130 may be configured to seal the opening 114 the reaction vial 110 of the vial device 100 (e.g., by a screw-fit of complementary threaded surfaces), such that the swab element 142 is inserted into the internal cavity 116 of the reaction vial 110, as depicted in FIG. 2B. The length of the base 144 and/or the swab stem 146 may enable the swab element 142 to extend into the internal cavity 116 and pierce one or both of the first and second frangible seals 120, 124, such that the sample, the reagent 122, and the liquid 118 may interact with each other in the internal cavity 116 to form a sample solution. The volume of the liquid 118 may be such that after reaction with the sample and the reagent 122 the sample solution does not reach the conduit 154 when the reaction vial 110 is in a vertical position, as shown in FIG. 1C. When the sample solution is ready for testing, the vial device 100 may be placed in a horizontal position, such that the cover 156 of the test and readout compartment 150 rests on a flat surface (e.g., a table), which may enable the sample solution to flow through the conduit 154 onto the LFA strip 180 in the testing chamber 152.

According to some embodiments of the present technology, a reagent may be added to the internal cavity 116 of the reaction vial 110 by a caged cap, as described in U.S. application Ser. No. 17/203,668 entitled “Reagent Carrier for Rapid Diagnostic Tests,” which is incorporated by reference herein. For example, instead of the reaction vial 110 of the vial device 100 including the reagent between the first and second frangible seals 120, 124, a reagent may be added to the reaction vial 110 via a cap that selectively releases the reagent into the internal cavity 116.

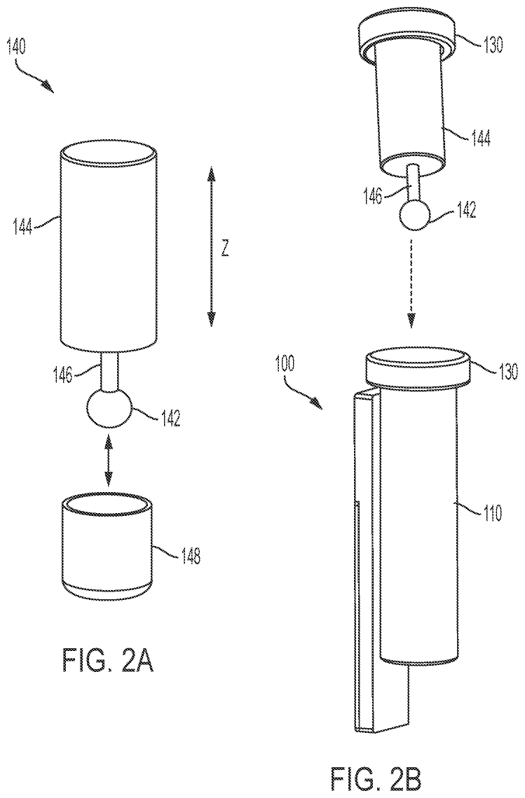

FIG. 3A shows an elevational side view of a caged cap 300 in a holding position according to some embodiments of the present technology, and FIG. 3B shows a perspective view of the caged cap 300 in a released position, In some embodiments, the caged cap 300 may be comprised of a cap base 310 supporting a retaining cage 320 configured to hold or confine a reagent 350 to the cap base 310, in the holding positions shown FIG. 3A. The retaining cage 320 may be comprised of a retainer base 321 and a plurality of fingers 322 extending from the retainer base 321 such that tips 322a of the fingers 322 are angled toward each other. In some embodiments, the cap base 310 may be formed of a hard material (e.g., a hard plastic, a metal, a wood-based material, and the like). In some embodiments, the retaining cage 320 may be formed of a resilient material (e.g., a synthetic rubber, a natural rubber, a silicone-based foam, and the like), which may be a material that flexes under application of a force but returns to an equilibrium form when no force is applied.

According to some embodiments of the present technology, when the caged cap 300 is in a rest state before the reagent 350 is loaded, the tips 322a may touch each other or may be spaced apart but nearly touch each other, such that there is a common space having a dimension that is smaller than a dimension of the reagent 350. When the retaining cage 320 is being loaded with the reagent 350, the resilient material forming the fingers 322 may flex outwards, away from each other, to accommodate the reagent 350 in the common space. After loading the reagent 350 into the common space, an internal restoring force in the resilient material may cause the tips 322a of the fingers 322 to try to return to their rest positions, thus imparting a holding force against the reagent 350 and causing the reagent 350 to be caged or held in place in the common space, in the holding position of the caged cap 300.

According to some embodiments of the present technology, the resilient material forming the retaining cage 320 may enable the retaining cage 320 to flex and move when the caged cap 300 is placed on the opening 114 of the reaction vial 110 and a force is applied by a user to attach the caged cap 300 to the reaction vial 110. Engagement of the reaction vial 110 with the caged cap 300 may cause movement and/or deformation of the fingers 322 of the retaining cage 320 sufficient to cause separation of the tips 322a of the fingers, thus enabling the reagent 350 to be released into the internal cavity 116 of the reaction vial 110. In some embodiments, the retaining cage 320 may be structured such that a surface of the retainer base 321 may come into contact with an edge or lip of the reaction vial 110 when the caged cap 300 is being attached to the reaction vial 110, thus providing the necessary contact and a release force F to cause movement and/or deformation of the fingers 322 to release the reagent 350 into the internal cavity 116. Such movement is schematically depicted in FIG. 3B by arrows representing flexing or movement of the fingers 322 in response to the release force F caused by movement and/or contact associated with attachment of the caged cap 300 to the reaction vial 110. The reagent 350 may fall into the internal cavity 116 due to gravity.

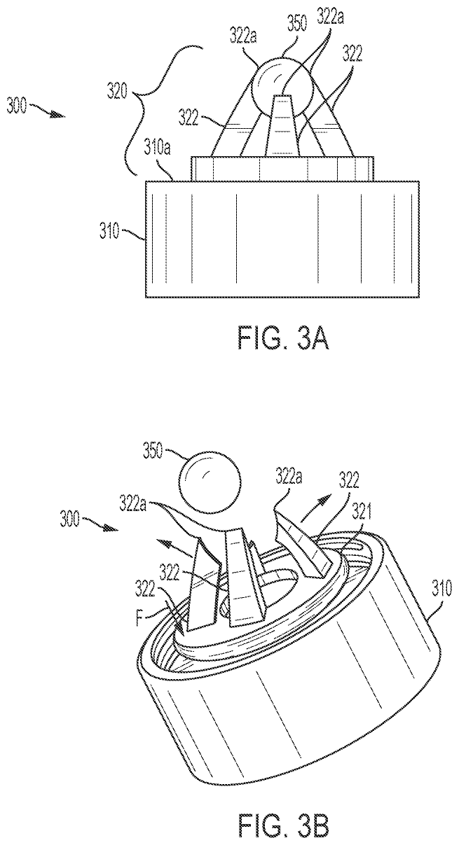

According to some embodiments of the present technology, the reaction vial 110 of the vial device 100 may be comprised of a rupturable ampoule 400, schematically shown in FIG. 4. The ampoule 400 may be comprised of an outer skin or shell 402 confining a liquid 404 within the ampoule 400. The liquid 404 may be, for example, any one or any combination of: a diluent liquid, a reagent solution (e.g., an amplification solution), and a buffer solution. The ampoule 400 may be configured to rupture when an external force applied to the outer skin or shell 402 exceeds a minimum force needed to compromise or break the outer skin or shell 402. In some embodiments, instead of or in addition to the liquid 118, the reaction vial 110 may be comprised of the rupturable ampoule 400. When the swab cap 130 of the swab device 100 is inserted in the reaction vial 110, the swab element 142 may extend into the internal cavity 116 and push on a surface of the rupturable ampoule 400 and pierce the outer skin or shell 402 of the ampoule 400 when fully inserted, enabling the liquid 404 to interact with the sample on the swab element to form a sample solution. Although the rupturable ampoule 400 is shown in FIG. 4 to be partially filled with the liquid 404, i.e., there is a space inside the outer skin or shell 402 that is not shown to contain the liquid 404, in some embodiments the liquid 404 may completely fill the space inside the outer skin or shell 402.

According to some embodiments, the outer skin or shell 402 of the rupturable ampoule 400 may be comprised of a polymer layer, or a metal foil layer, or a multilayer structure that includes both a polymer layer and a metal foil layer. The polymer layer may be comprised of any one or any combination of: a polyvinyl chloride (PVC) layer and a low-density polyethylene (LDPE) layer. In some embodiments, a thickness of the outer skin or shell 402 may be in a range of 2 mil to 20 mil, or 4 mil to 15 mil, or 5 mil to 10 mil. Although the rupturable ampoule 400 is depicted in FIG. 4 to have a shape of a capsule, it should be understood that other shapes may be used. For example, in some embodiments the outer skin or shell 402 may be a bag or an envelope or a pod in which the liquid 404 is confined. In some embodiments, a rupture force for breaking the outer skin or shell 402 may be any one or any combination of: a pinching force, a squeezing force, a compressive force, a tearing force, a twisting force, a frictional force, and an impaling or piercing force. A value of the rupture force may be between 0.2 pound and 1.5 pounds, or between 1 pound and 2 pounds, or between 0.5 pound and 3 pounds.

According to some embodiments of the present technology, a test kit (not shown) may be comprised of the swab device 140 and the vial device 100. The test kit may be a stand-alone kit useable by a person who is not a medical professional to obtain a sample (e.g., with the swab device 140), to form a sample solution by interacting the sample with one or more reagents and/or one or more fluids (e.g., with the reaction vial 110 of the vial device 100), to interact the sample solution with an assay vehicle such as an LFA strip (e.g., with the test and readout device 150 of the vial device 100), to allow test results to be read (e.g., via the window 160 of the test and readout device 150). As will be appreciated, the test kit also may include instructions (e.g., a paper guide, a link to a website, a CD, etc.) and/or one or more reagents (e.g., reagents releasably held in caged caps).

2.2 Vial Device with Rupturable Ampoule

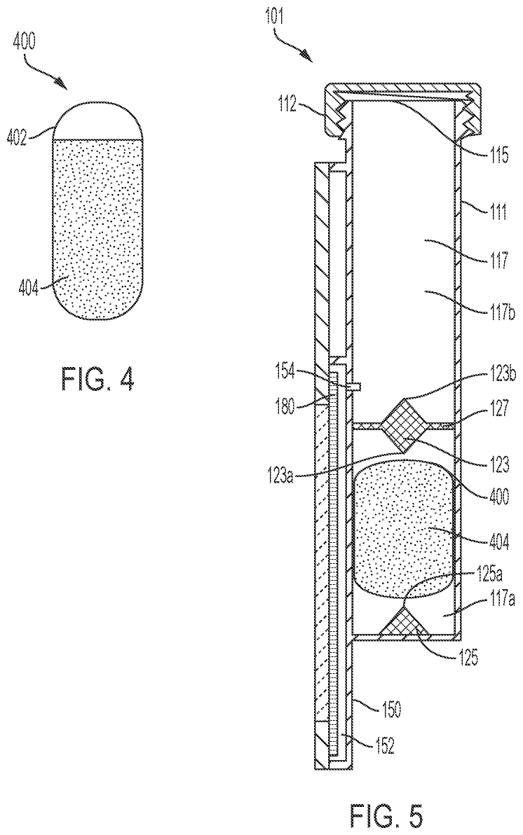

FIG. 5 depicts an elevational view of a cross section of a vial device 101, according to some embodiments of the present technology. In some embodiments, features of the vial device 101 that are the same as or similar to those of the vial device 100 described above will have the same reference numerals and will not be described separately. The vial device 101 may be a rupturable diagnostic device because a rupturing procedure may occur during ordinary use of the vial device 101, as discussed below. According to some embodiments, the vial device 101 may be comprised of a reaction vial 111 and a test and readout device 150 (described above). The reaction vial 111 may be comprised of an internal cavity 117 in which a sample may interact with one or more reagents. During storage, an opening 115 of the internal cavity 117 may be covered by a removable cap 112.

According to some embodiments of the present technology, the internal cavity 117 of the reaction vial 111 may be comprised of a first chamber 117a, a second chamber 117b, a rupturable ampoule 400 containing a liquid (e.g., a reagent fluid), and a first rupturing object 123 separating the first chamber 117a from the second chamber 117b. Optionally, in some embodiments, the reaction vial 111 may be comprised of a second rupturing object 125 located at a bottom end of the first chamber 117a. In some embodiments, the first rupturing object 123 may be suspended between the first and second chambers 117a, 117b by an open structure 127, such that fluid may flow freely between the first and second chambers 117a, 117b. The structure 127 may be formed of an elastic material (e.g., rubber), enabling the structure 127 to grip a surface of the internal cavity 117 during storage or during transit of the vial device 101, and also enabling the structure 127 to stretch and move when a force is applied. Alternatively, the structure 127 may be formed of a rigid material and may be slidably mounted in the internal cavity 117 between a storage position, where little or no force is applied to the rupturable ampoule 400, and a piercing position, where a piercing force sufficient to rupture the rupturable ampoule 400 is applied. In some embodiments, the structure 127 and the first and second rupturing objects 123, 125 may be formed of (or coated with) a material that is inert to reagents and reagent solutions that may be held in the internal cavity during a diagnostic test.

According to some embodiments of the present technology, the first rupturing object 123 may be configured to have a first sharp point or edge 123a facing into the first chamber 117a toward the rupturable ampoule 400. Optionally, the first rupturing object 123 may have a second sharp point or edge 123b facing into the second chamber 117b. The second rupturing object 125 (if present) may be configured to have a sharp point or edge 125a facing into the first chamber 117a toward the rupturable ampoule 400. In some embodiments, the rupturable ampoule 400 may be configured to be wedged in the first chamber 117a between the first sharp point or edge 123a on the first rupturing object 123 and the sharp point or edge 125a on the second rupturing object 125. In some embodiments, the rupturable ampoule 400 may sit on or be wedged at or near the bottom end of the first chamber 117a.

According to some embodiments of the present technology, the vial device 101 may be used with the swab device 140 (see FIG. 2A). For example, during a diagnostic test, a reagent may be present in the internal cavity 117 (e.g., resting on the structure 127 and/or included in the rupturable ampoule 400) or may be added to the internal cavity 117 using a caged cap (e.g., the caged cap 300 of FIG. 3A). The swab element 142 of the swab device 140, bearing a sample to be tested, may be inserted into the internal cavity 117 and may push the first rupturing object 123 and/or the structure 127 to cause the first sharp point or edge 123a on the first rupturing object 123 and/or the sharp point or edge 125a on the second rupturing object 125 to contact and pierce the rupturable ampoule 400. After rupturing, the liquid 404 in the rupturable ampoule 400 may interact with the sample (and with an added reagent, if applicable) to form a sample solution. As with the vial device 100, the volume of the liquid 404 may be such that after reaction with the sample, a level of the sample solution may not reach the conduit 154 when the reaction vial 111 is in a vertical position. When the sample solution is ready for testing, the vial device 101 may be placed in a horizontal position such that the cover 156 of the test and readout compartment 150 may rest on a flat surface, which may enable the sample solution to flow through the conduit 154 onto the LFA strip 180 in the testing chamber 152.

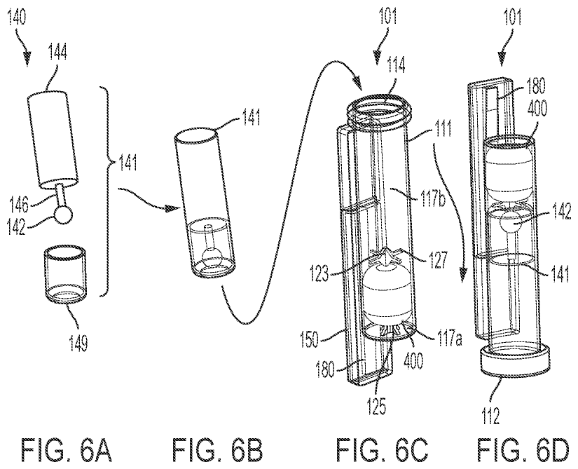

According to some embodiments of the present technology, the vial device 101 may be used with a pierceable container 141 configured to rupture during a test procedure. FIGS. 6A through 6D depict a flow sequence of acts that may be performed to load the vial device 101 with a sample contained in the pierceable container 141, according to some embodiments. In some embodiments, the pierceable container 141 may be comprised of the swab device 140 and a pierceable vial 149. The pierceable vial 149 may be formed of a material that may be pierced with the first rupturing object 123. In some embodiments, the pierceable vial 149 may be formed of a polymeric material. For example, the polymeric material may be comprised of any one or any combination of: polyethylene terephthalate (PET), high-density polyethylene (HDPE), and polypropylene (PP). As will be appreciated, the pierceable material need not be polymeric and, in other embodiments, may be another material (e.g., a pierceable metal-based foil, a pierceable glass container, a paper or cardboard-based material lined with a polymeric material and/or a metal-based material impervious to liquids, etc.) that may be intentionally ruptured or broken with the first rupturing object 123.

According to some embodiments of the present technology, the pierceable vial 149 may contain a solution configured to interact with the sample on the swab element 142. For example, the pierceable vial 149 may contain a lysis solution and/or an amplification solution configured to promote lysis and to amplify a target species resulting from the lysis. In some embodiments, as depicted in FIG. 6A, the swab element 142 bearing the sample may be inserted in a cavity of the pierceable vial 149. As depicted in FIG. 6B, the pierceable vial 149 may be joined with the base 144 of the swab device 140 after the swab element 142 has been inserted. The sample may form a sample fluid with the solution in the pierceable vial 149. A leak-tight seal may be formed between the base 144 and the pierceable vial 149, confining the sample fluid within the cavity of the pierceable vial 149, according to some embodiments.

According to some embodiments of the present technology, as depicted in FIG. 6C, the pierceable container 141 containing the sample fluid may be inserted through the opening 114 of the vial device 101 into the second chamber 117b, with the vial device 101 being in an upright position such that the opening 114 faces upwards (i.e., opposite to the direction of the force of gravity). After the pierceable container 141 has been inserted into the second chamber 117b, the opening 114 may be sealed with the cap 112. In some embodiments, the pierceable container 141 may be sized to have a length such that, when the pierceable container 141 is seated in the second chamber 117b so that the opening 114 is able to be sealed with the cap 112, the pierceable vial 149 exerts a rupture force against the first rupturing object 123. The rupture force may be a force sufficient to cause the first rupturing object 123 to push against the rupturable ampoule 400 to squeeze the rupturable ampoule 400 against the first sharp point or edge 123a on the first rupturing object 123 and, if the second rupturing object 124 is present, against the sharp point or edge 125a on the second rupturing object 125, to rupture the rupturable ampoule 400. Upon rupturing, the liquid 404 in the rupturable ampoule 400 may be released into the first chamber 117a. The rupture force also may be a force sufficient to cause the second sharp point or edge 123b on the first rupturing object 123 to push against the pierceable container 141 to pierce the pierceable vial 149. Upon piercing, the sample fluid in the pierceable vial 149 may be released into the second chamber 117b and allowed to flow into the first chamber 117a under the force of gravity. In some embodiments, the liquid 404 in the rupturable ampoule 400 may be a diluent fluid 404 configured to dilute the sample fluid to form a diluted sample fluid in the first chamber 117a.

In some embodiments of the present technology, a total volume of the sample fluid and the diluent fluid 404 may be such that the diluted sample fluid may be unable to reach the conduit 154 connecting the testing compartment 152 with the internal cavity 117 of the reaction vial 111 when the reaction vial 111 is in the upright position. Such a structural feature may be used advantageously to prevent the LFA strip 180 in the testing compartment 152 from coming into contact with the diluted sample fluid until a predetermined amount of mixing time (e.g., 30 seconds, one minute, five minutes, etc.) has elapsed, to enable the sample fluid to mix or interact with the diluent fluid 404. After the predetermined amount of mixing time has passed, the vial device 101 may be inverted to an upside-down position, as shown FIG. 6D, in which the cap 112 may face downward, or the vial device 101 may be placed in a horizontal position in which the cover 156 of the test and readout compartment 150 rests on a flat surface, which may enable the diluted sample fluid to flow through the conduit 154 onto the LFA strip 180 in the testing chamber 152.

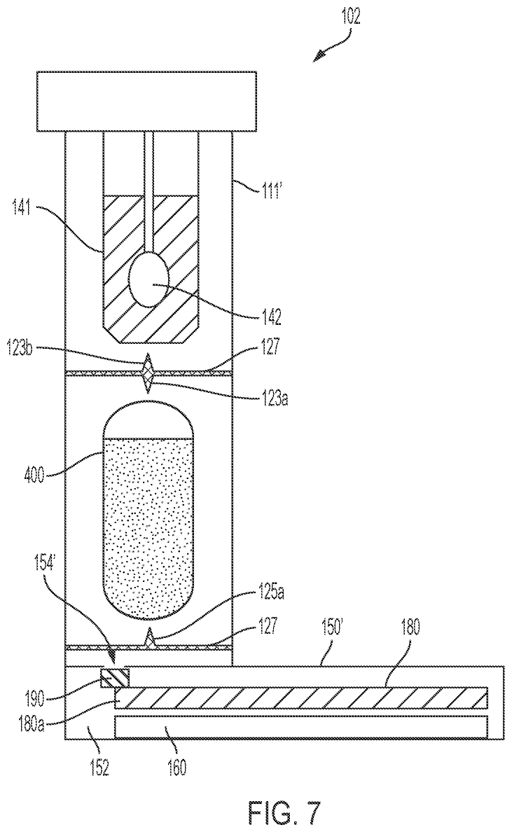

According to some embodiments of the present technology, a vial device 102 may be a variation of the vial device 101. Unlike the vial device 101, in which the conduit 154 is located in a mid-section of the internal cavity 117, a conduit 154′ of the vial device 102 may be located at or near the bottom end of the first chamber 117a, as depicted in FIG. 7. In some embodiments, the vial device 102 may be comprised of a reaction vial 111′ that is similar to the reaction vial 111 except for the location of the conduit 154′. The vial device 102 also may be comprised of a test and readout device 150′, which may be a variation of the test and readout device 150. In some embodiments, the test and readout device 150′ may be configured such that the conduit 154′ is proximate an intake end 180a of a LFA strip 180, as shown in FIG. 7. A sample pad 190 may be attached to the intake end 180a of the LFA strip 180 and may be located between the conduit 154′ and the intake end 180a of the LFA strip 180. With such an arrangement, the diluted sample fluid may be absorbed by the sample pad 190 after passing through the conduit 154′ and before coming into contact with the LFA strip 180. In some embodiments, the sample pad 190 may dampen the intake end 180a of LFA strip 180 with the diluted sample fluid, and the LFA strip 180 may subsequently wick the diluted sample fluid from the sample pad 190 by capillary action. The sample pad 190 may operate to retard or delay the movement of the diluted sample fluid across the LFA strip 180 by limiting a flow rate of the diluted sample fluid such that the movement is wholly or predominantly via wicking or capillary action. As will be appreciated, although the vial device 102 is shown FIG. 7 to have the reaction vial 111′ oriented at a right angle to the test and readout device 150′, in some embodiments the reaction vial 111′ may be oriented parallel to the test and readout device 150′ or another angle relative to the test and readout device 150′, provided that the conduit 154′ between the internal cavity 117 of the reaction vial 111′ and the test and readout device 150′ causes the diluted sample fluid to be introduced to the LFA strip 180 at the intake end 180a and via the sample pad 190.

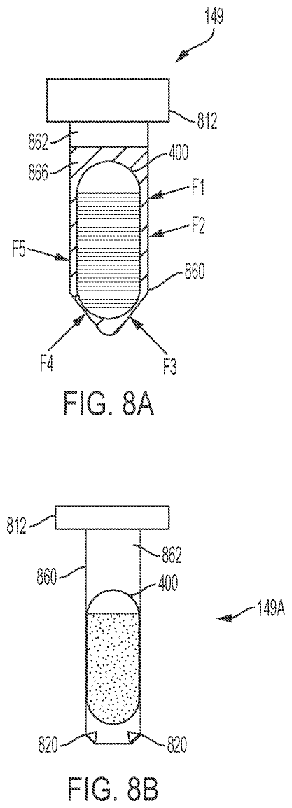

According to some embodiments of the present technology, the pierceable vial 149 may have a configuration shown in FIG. 8A. Prior to use in a test procedure (e.g., during storage), the pierceable vial 149 may be comprised of a container 860 holding a liquid 866 in an internal cavity 862 of the container 860. A cap 812 may confine the liquid 866 to the internal cavity 862. In some embodiments, when used with the swab device 140, the container 860 may be configured to form a leak-tight seal with the base 144 of the swab device 140 after the swab element 142 has been inserted, as discussed above. In some embodiments, the container 860 may be formed of a resilient material, such that a shape of the container 860 may be deformed elastically by application of an external force and, when the external force is no longer being applied, the container 860 may return to its pre-deformation shape (or to nearly its pre-deformation shape) without breaking or rupturing. In some embodiments, the container 860 may ruptured by being pierced with a sharp object (e.g., a pin, a spike, a sharp edge, a hook, a serrated edge, an abrasive surface, the rupturing object 123, etc.). In some embodiments, in addition to or instead of the liquid 866, the internal cavity 862 of the container 860 may hold a rupturable ampoule 400, which may burst when the swab element 142 is inserted in the internal cavity 862 and/or when an external force is applied to the container 860 to deform the container 860. For example, the external force may be any one or any combination of forces F1, F2, . . . , F5 applied at one or more locations on an external surface of the container 860. In some embodiments, the external force may have a value between 0.2 pound and 1.5 pounds, or between 1 pound and 2 pounds, or between 0.5 pound and 3 pounds. The elastic deformation produced by the external force may be any one or any combination of: a bending deformation, a pinching deformation, and a twisting deformation. The rupturable ampoule 400 held in the container 860 may rupture when at least some of the external force applied to the container 860 is transferred to the outer skin or shell 402 of the rupturable ampoule 400 and exceeds a rupture force, which may be a minimum force needed to compromise or break the outer skin or shell 402.

According to some embodiments of the present technology, the pierceable vial 149 may be comprised of the rupturable ampoule 400 but not the liquid 866. In some embodiments, the liquid 404 in the rupturable ampoule 400 may be comprised of a lysis solution and/or an amplification solution. In some embodiments, when a sample is inserted in the internal cavity 862 of the container 860 via the swab element 142 of the swab device 140, and when the outer skin or shell 402 of the rupturable ampoule 400 is ruptured, the lysis and/or amplification solution may interact with a sample to form a sample solution contained in the container 860 of the pierceable vial 149. The pierceable vial 149 may then be inserted in the vial device 101, 102, as discussed above. In some embodiments, the rupturable ampoule 400 in the vial device 101, 102 may be a diluent fluid configured to dilute the sample solution to form a diluted sample fluid. Optionally, prior to insertion in the vial device 101, 102, the pierceable vial 149 containing the sample solution maybe heated with a heater to promote lysis and/or to promote an increased degree of amplification. As will be appreciated, although the rupturable ampoule 400 is depicted in FIG. 8A to be spaced apart from an internal surface of the container 860, it should be understood that the rupturable ampoule 400 may be sized to fit snugly in the container 860 such that one or more portions touch the internal cavity 862, and may even conform in shape to a shape of the internal cavity 862.

According to some embodiments of the present technology, the pierceable vial 149 may be comprised of the liquid 866 and the rupturable ampoule 400, as depicted in FIG. 8A. In some embodiments, the liquid may be a buffer solution configured to activate a lyophilized reagent. In some embodiments, the lyophilized reagent may be added to the container 860 via a caged cap (e.g., the caged cap 300), as discussed above. For example, the lyophilized reagent may be comprised of a lysis reagent and an amplification reagent that both may be activated by the liquid 866 to form a reagent solution. In some embodiments, when a sample is introduced to the reagent solution via the swab device 140, a sample solution may be formed. In some embodiments, the liquid 404 in the rupturable ampoule 400 may be a diluent fluid. When the rupturable ampoule 400 is ruptured via deformation of the container 860, the sample solution may be diluted by the diluent fluid to form a diluted sample solution. Optionally, prior to deformation of the container 860, the sample solution maybe heated with a heater to promote lysis and/or an increased degree of amplification. When the diluted sample solution is ready for testing, the container 860 may be pierced to allow the diluted sample solution to contact an assay vehicle (e.g., a LFA strip).

According to some embodiments of the present technology, a pierceable vial 149A may have a configuration shown in FIG. 8B and may be similar to the pierceable vial 149 except for a presence of one or more piercing objects 820 in the internal cavity 862. The piercing objects 820 may come into contact with the rupturable ampoule 400 when the container 860 undergoes deformation. For example, the piercing objects 820 may be comprised of one or more tacks, which may protrude from a side surface or a bottom surface or a corner of the internal cavity 862 but may not touch the rupturable ampoule 400 until the container 860 is crushed or deformed. The piercing objects 820 may advantageously enable the outer skin or shell 402 to be thicker and sturdier and less susceptible to being ruptured by accidental squeezing of the container 860. In some embodiments, when the container 860 of the pierceable vial 149A is crushed by application of an external force, deformation of the container 860 may cause the piercing objects 820 to come into contact with and pierce the outer skin or shell 402 and cause the rupturable ampoule 400 to rupture.

2.3 Crush Effector with Crushable Ampoule

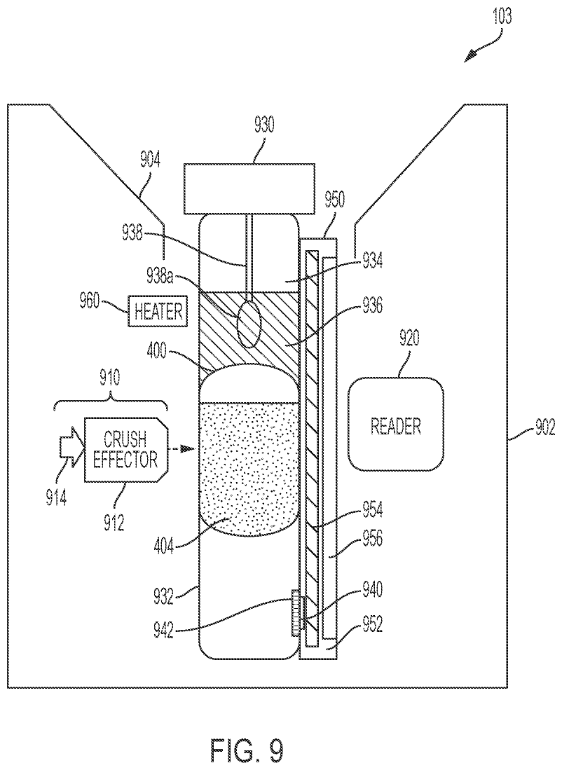

FIG. 9 depicts an elevational view of a cross section of diagnostic device comprised of a crush-type vial device (“crusher device”) 103, according to some embodiments of the present technology. As with the other vial devices 101, 102, the crusher device 103 is comprised of at least one rupturable component configured to rupture or break during a test procedure, to enable reagents to intermingle before undergoing testing with an assay vehicle (e.g., an LFA strip). In some embodiments, the crusher device 103 may be comprised of a housing 902, an electromechanical crushing device 910 disposed in the housing 902, an electronic reader 920 disposed in the housing 902, and a test vial 930. Optionally, in some embodiments, the crusher device 103 may be comprised of a heater 960 disposed in the housing. The housing 902 may have a recessed port 904 configured to receive the test vial 930 therein. In some embodiments, the test vial 930 may be removable from the recessed port 904. In some embodiments, insertion of the test vial 930 into the recessed port 904 may activate the heater 960 to automatically heat the test vial 930 according to a pre-programmed temperature profile.

According to some embodiments of the present technology, the test vial 930 may be comprised of a reaction vial 932 attached to a test and readout device 950. The reaction vial 932 may be comprised of a container formed of a deformable material that may also be resilient, such that a shape of the container may be deformed elastically by application of an external force and, when the external force is no longer being applied, the container may return to its pre-deformation shape (or to nearly its pre-deformation shape) without breaking or rupturing, similar to the container 860 described above. In some embodiments, the reaction vial 932 may be comprised of a rupturable ampoule 400 containing a liquid 404 (e.g., a diluent fluid), such as described above. In some embodiments, the rupturable ampoule 400 may be lodged snugly in a mid-section of an internal cavity 934 of the reaction vial 932. The reaction vial 932 also may be comprised a fluid 936 confined to a region of the internal cavity 934 above the rupturable ampoule 400. In some embodiments, prior to the reaction vial 932 being used for diagnostic testing (e.g., during storage), the internal cavity 934 may be sealed with a leak-tight storage cap, with the rupturable ampoule 400 and the fluid 936 held in the internal cavity 934. In some embodiments, during a test procedure for a diagnostic test, the storage cap may be removed to allow a swab device 938 bearing a sample to be inserted. In some embodiments, the swab device 938 may have an integrated cap configured to seal the internal cavity 934 with a swab element 938a in contact with the fluid 936, as shown in FIG. 9. In some other embodiments, the swab device 938 may not have an integrated cap but may fit entirely in the internal cavity 934 such that it may be permitted to remain in the internal cavity 934 while the storage cap is used to reseal the internal cavity 934. In some embodiments, the swab device 938 may be used to add the sample to the fluid 936 (e.g., by swirling the swab element 938a in the fluid 936) and then the swab device 938 may be discarded.

According to some embodiments of the present technology, the fluid 936 may be comprised of a lysis reagent that, when exposed to the sample on the swab element 938a, causes the sample to undergo a lysis reaction. In some embodiments, the fluid 936 may be comprised of an amplification reagent that may amplify a result of the lysis reaction. A sample fluid may result from an interaction of the sample with the fluid 936. In some embodiments, the heater 960 may be used to accelerate lysis and/or amplification.

The test and readout device 950 may be similar to the test and readout device 150′ (see FIG. 7) and therefore will not be described in detail. In some embodiments, a conduit 940 may connect the internal cavity 934 of the reaction vial 932 with a testing compartment 952 of the test and readout device 950. A sample pad 942 may be positioned at the conduit 940 in the reaction vial 932, as shown in FIG. 9, or in the testing compartment 952. in some embodiments, the sample pad 942 may cover the conduit 940 to ensure that the sample fluid passes through the sample pad 942 to enter the testing compartment 952. The test and readout device 950 may be comprised of an LFA strip 954 disposed in the testing compartment 952 such that an intake end of the LFA strip 954 is proximate the conduit 940, as shown in FIG. 9. In some embodiments in which the sample pad 942 is disposed in the testing compartment 952, the intake end of the LFA strip 954 may be in contact with the sample pad 942.

According to some embodiments of the present technology, the electromechanical crushing device 910 may be comprised of a crush effector 912 and an actuator 914. In some embodiments, movement of the crush effector 912 may be controlled by the actuator 914. For example, the crush effector 912 may be comprised of a pincher configured to apply a pinching force to the reaction vial 932 to cause deformation to the reaction vial 932 and, consequently, to cause the rupturable ampoule 400 to burst or rupture. The liquid 404 in the rupturable ampoule 400 may, in some embodiments, be a diluent liquid. Upon rupturing, the diluent liquid 404 may interact with sample fluid (formed from the sample and the liquid 936, discussed above) and may mix in the internal cavity 934 to form a diluted sample fluid. In some embodiments, the force of gravity may cause the diluted sample fluid to flow to a region near the conduit 940 where the diluted sample fluid may be absorbed by the sample pad 942. In turn, the diluted sample fluid absorbed by the sample pad 942 may dampen the intake end the LFA strip 954 and, via capillary action, the diluted sample fluid may interact with test substances on the LFA strip 954 downstream from the intake end. In some embodiments, a soluble layer (not shown) may coat the sample pad 942. The soluble layer (e.g., a pectin layer) may be configured to dissolve in the diluted sample fluid, and to be inert to the diluted sample fluid and inert to the test substances on the LFA strip 954 when dissolved. In some embodiments, the soluble layer may be used to delay movement of the diluted sample fluid to the LFA strip 954. For example, the soluble layer may be configured to dissolve in an amount of time (e.g., 30 seconds, one minute, two minutes, five minutes, etc.) sufficient to enable mixing of the diluent liquid 404 and the sample fluid after the rupturable ampoule 400 has been ruptured.

According to some embodiments of the present technology, the test and readout device 950 may be transparent or may have a transparent window 956, permitting test regions on the LFA strip 954, such as regions where the test substances are located, to be visible and read by the electronic reader 920. In some embodiments, the electronic reader may be comprised of a camera configured to capture an image of the test regions of the LFA strip 954 and to provide image data to a remote display device and/or to an computer processor programmed with an algorithm for analyzing the image to detect features indicative of a pathogen. In some embodiments, when the test vial 930 is seated in the recessed port 904 of the crusher device 103, the window 956 may be positioned opposite the electronic reader 920.

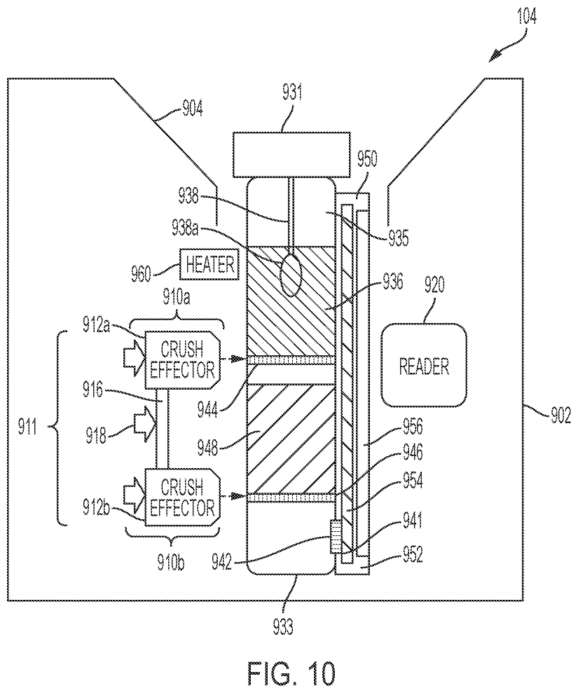

FIG. 10 depicts an elevational view of a cross section of diagnostic device comprised of another crusher device 104, according to some embodiments of the present technology. The crusher device 104 is in many ways similar to the crusher device 103 and therefore the following discussion will point out notable differences without describing features that are the same.

According to some embodiments of the present technology, the crusher device 104 may be comprised of a housing 902, an electromechanical crushing device 911 disposed in the housing 902, an electronic reader 920 disposed in the housing 902, and a test vial 931. Optionally, in some embodiments, the crusher device 104 may be comprised of a heater 960 disposed in the housing.

According to some embodiments of the present technology, the test vial 931 may be comprised of a reaction vial 933 attached to a test and readout device 950. The reaction vial 933 may be comprised of a container formed of a deformable material that also may be resilient. In some embodiments, the reaction vial 933 may be comprised of a first frangible seal 944 and a second frangible seal 946. The first frangible seal 944 may be configured to confine a first fluid 936 to an upper portion of an internal cavity 935 of the reaction vial 933, relatively closer to an opening of the internal cavity 935. The second frangible seal 946 may be configured to confine a second fluid 948 to a mid-portion of the internal cavity 935, which may be farther from the opening of the internal cavity 935 relative to the first frangible seal 944. A conduit 941 may connect a lower portion of the internal cavity 935 to the test and readout device 950.

According to some embodiments of the present technology, the fluid 936 may be comprised of a lysis reagent that, when exposed to a sample on a swab element 938a of a swab device 938, causes the sample to undergo a lysis reaction. In some embodiments, the fluid 936 may be comprised of an amplification reagent that may amplify a result of the lysis reaction. A sample fluid may result from an interaction of the sample with the fluid 936. In some embodiments, the heater 960 may be used to accelerate lysis and/or amplification. In some embodiments, the first frangible seal 944 may be a leak-tight seal that may enable the sample fluid to be prepared in the upper portion of the internal cavity 935 and confined to the upper portion of the internal cavity 935 until the first frangible seal 944 is ruptured, as discussed below. In some embodiments, the second fluid 948 confined to the mid-portion of the internal cavity 935 may be a diluent fluid configured to dilute the sample fluid.

As noted above, the conduit 941 may connect the lower portion of the internal cavity 935 to the test and readout device 950. According to some embodiments of the present technology, a sample pad 942 may be positioned at the conduit 941 in the reaction vial 933, or in a testing compartment 952 of the test and readout device 950, or may have a portion in the reaction vial 933 and a portion in the testing compartment 952, as shown in FIG. 10. The test and readout device 950 may be comprised of an LFA strip 954 disposed in the testing compartment 952 such that an intake end of the LFA strip 954 is proximate the conduit 941, as shown in FIG. 10, and may, in some embodiments, be in contact with the sample pad 942.

According to some embodiments of the present technology, the electromechanical crushing device 911 may be comprised of a first crusher 910a and a second crusher 910b. In some embodiments, the first crusher 910a may be the same as the electromechanical crushing device 910 described above and may be comprised of a crush effector 912a configured to deform the reaction vial 933 at or near a location of the first frangible seal 944, to cause the first frangible seal 944 to rupture. In some embodiments, upon rupturing of the first frangible seal 944, the sample fluid may interact with the diluent fluid 948 to form a diluted sample fluid. In some embodiments, the diluted sample fluid may be prevented from reaching the lower portion of the internal cavity 935 by the second frangible seal 946. In some embodiments, the second crusher 910b may be the same as the electromechanical crushing device 910 described above and may be comprised of a crush effector 912b configured to deform the reaction vial 933 at or near a location of the second frangible seal 946, to cause the second frangible seal 946 to rupture. In some embodiments, upon rupturing of the second frangible seal 946, the diluted sample fluid may flow into the lower portion of the internal cavity 935 and wet the sample pad 942. As will be appreciated, the first and second crushers 910a, 910b may work independently of each other to, for example, selectively bend the reaction vial 933 to break only the first frangible seal 944 or to selectively bend the reaction vial 933 to break only the second frangible seal 946.

For some diagnostic tests, it may be advantageous to break the first and second frangible seals 944, 946 simultaneously or nearly simultaneously. According to some embodiments of the present technology, the electromechanical crushing device 911 may be comprised of a structure 916 (e.g., a bar) configured to lock the crush effectors 912a, 912b of the first and second crushers 910a, 910b together, and also may be comprised of an actuator 918 configured to control movement of the crush effectors 912a, 912b. In some embodiments, the actuator 918 may control the crush effectors 912a, 912b to apply a steady force against the reaction vial 933 at the locations of the first and second frangible seals 944, 946, to deform the reaction vial 933 and thereby rupture the first and second frangible seals 944, 946. In some other embodiments, the crush effectors 912a, 912b may operate like reciprocal hammers under control of the actuator 918, such that a repetitive force may be applied against the reaction vial 933 at the locations of the first and second frangible seals 944, 946, to rupture the first and second frangible seals 944, 946.

### 3. Test Methodologies

The diagnostic devices described herein may be used to detect whether a test subject is afflicted with a communicable disease by detecting whether a target nucleic-acid sequence corresponding to a pathogen of interest and indicative of the disease is present in a sample obtained from the test subject. The sample may be comprised of, for example, saliva and/or mucus obtained from the test subject, and/or may be cells obtained from the test subject by other means (e.g., by scraping the test subject's skin). Target nucleic-acid sequences and techniques that may be used for their detection are described below.

Target nucleic-acid sequences may be associated with a variety of diseases or disorders. In some embodiments of the present technology, the diagnostic devices described herein may be used to diagnose at least one disease or disorder caused by a pathogen. In some embodiments, the diagnostic devices may be configured to detect a nucleic acid encoding a protein (e.g., a nucleocapsid protein) of SARS-CoV-2, which is the virus that causes COVID-19. In some embodiments, the diagnostic devices may be configured to identify particular strains of a pathogen (e.g., a virus). In some embodiments, a diagnostic device may utilize and be comprised of an assay vehicle (e.g., an LFA strip) comprised of a first test line configured to detect a nucleic-acid sequence of SARS-CoV-2 and a second test line configured to detect a nucleic-acid sequence of a SARS-CoV-2 virus having a D614G mutation (i.e., a mutation of the 614^{th} amino acid from aspartic acid (D) to glycine (G)) in its spike protein. In some embodiments, one or more target nucleic-acid sequences may be associated with a single-nucleotide polymorphism (SNP). In certain cases, the diagnostic devices may be used for rapid genotyping to detect whether a SNP, which may affect medical treatment, is present.

In some embodiments of the present technology, the diagnostic devices described herein may be configured to diagnose two or more diseases or disorders. This may be referred to herein as multiplexed testing. In certain cases, for example, a diagnostic device may utilize and be comprised of an LFA strip comprised of a first test line configured to detect a nucleic-acid sequence of SARS-CoV-2, a second test line configured to detect a nucleic-acid sequence of an influenza virus (e.g., an influenza A virus), and a third line configured to detect a nucleic-acid sequence of another influenza virus (e.g., an influenza B virus) or a nucleic acid sequence of a bacterium.

3.1 Lysis of Samples

According to some embodiments of the present technology, lysis may be performed on a sample by chemical lysis techniques (e.g., exposing the sample to one or more lysis reagents) and/or thermal lysis techniques (e.g., heating the sample). In chemical lysis, lysis may be performed by one or more lysis reagents, discussed below.

According to some embodiments of the present technology, a lysis reagent may be in solid form (e.g., lyophilized, dried, crystallized, air jetted, etc.). For example, a solid lysis reagent may be in the form of a pellet, or capsule, or gelcap, or tablet. In some embodiments, a solid lysis reagent may be included in a caged cap, as described above. In some embodiments, a lysis reagent may be comprised of one or more additional reagents (e.g., a reagent to reduce or eliminate cross contamination).

According to some embodiments of the present technology, a solid lysis reagent may be shelf stable for a relatively long period of time. In some embodiments, a lysis pellet, or capsule, or gelcap, or tablet may be shelf stable for at least 1 month, at least 3 months, at least 6 months, at least 1 year, at least 5 years, or at least 10 years. In some embodiments, a solid lysis reagent may be thermostabilized and may be stable across a wide range of temperatures. In some embodiments, a lysis pellet, or capsule, or gelcap, or tablet may be stable at a temperature of at least 0° C., at least 10° C., at least 20° C., at least 37° C., at least 65° C., or at least 100° C. As will be appreciated, a solid lysis reagent may be activated before or during use with a sample by contact with a buffer fluid.

As noted above, thermal lysis may be accomplished by applying heat to a sample. According to some embodiments of the present technology, thermal lysis may be performed by applying a lysis heating protocol comprised of heating the sample at one or more temperatures for one or more time periods or durations using any suitable heater (e.g., the heater 960).

3.2 Nucleic-Acid Amplification

Following lysis, one or more target nucleic acids (e.g., a nucleic acid of a target pathogen) may be amplified, according to some embodiments of the present technology. In some embodiments, DNA may be amplified according to any nucleic-acid amplification method known in the art. For example, nucleic-acid amplification methods that may be employed may include isothermal amplification methods, which include: loop-mediated isothermal amplification (LAMP), recombinase polymerase amplification (RPA), nicking enzyme amplification reaction (NEAR), thermophilic helicase dependent amplification (tHDA), nucleic acid sequence-based amplification (NASBA), strand displacement amplification (SDA), isothermal multiple displacement amplification (IMDA), rolling circle amplification (RCA), transcription mediated amplification (TMA), signal mediated amplification of RNA technology (SMART), single primer isothermal amplification (SPIA), circular helicase-dependent amplification (cHDA), whole genome amplification (WGA), and CRISPR-related amplification, such as CRISPR-Cas9-triggered nicking endonuclease-mediated strand displacement amplification (CRISDA). In some embodiments, an isothermal amplification method that may be performed in a test procedure may be comprised of applying heat to a sample. For example, heat may be applied to a sample fluid containing the sample. In some embodiments, the isothermal amplification method may be comprised of applying an amplification heating protocol, which may be comprised of heating the sample at one or more temperatures for one or more time periods using any appropriate heater (e.g., the heater 960).

In embodiments where a target pathogen may have RNA as its genetic material, the target pathogen's RNA may need to be reverse transcribed to DNA prior to amplification.

3.3 Molecular Switches

As described herein, a sample may undergo lysis and amplification prior to detection of a target nucleic-acid sequence. Reagents associated with lysis and/or amplification may be in solid form (e.g., lyophilized, dried, crystallized, air jetted, etc.). According to some embodiments of the present technology, one or more (and, in some cases, all) of the reagents necessary for lysis and/or amplification may be present in a single pellet, capsule, gelcap, or tablet. In some embodiments, the pellet, capsule, gelcap, or tablet may be comprised of two or more enzymes, and it may be necessary for the enzymes to be activated in a particular order. Therefore, in some embodiments, the enzyme-containing tablet, pellet, capsule, or gelcap may further be comprised of one or more molecular switches.

Molecular switches, as used or described herein, may be molecules that, in response to certain conditions, reversibly switch between two or more stable states. According to some embodiments of the present technology, a condition that causes a molecular switch to change its configuration may be associated with any one or any combination of: pH, light, temperature, an electric current, microenvironment, and presence of ions and/or other ligands. In some embodiments, the condition may be heat. In some embodiments, the molecular switches may be comprised of aptamers. Aptamers may refer generally to oligonucleotides or peptides that may bind to specific target molecules (e.g., the enzymes described herein). The aptamers, upon exposure to heat or other conditions, may dissociate from the enzymes. With use of molecular switches, one or more of the processes described herein (e.g., lysis, decontamination, reverse transcription, amplification, etc.) may be performed in a single test tube with a single enzymatic tablet, pellet, capsule, or gelcap.

3.4 CRISPR/Cas Techniques

According to some embodiments of the present technology, CRISPR/Cas detection techniques may be used to detect a target nucleic-acid sequence. For example, one or more CRISPR/Cas detection reagents may be included on an LFA strip. CRISPR generally may refer to Clustered Regularly Interspaced Short Palindromic Repeats, and Cas generally may refer to a particular family of proteins. In some embodiments, a CRISPR/Cas detection platform or technique may be combined with an isothermal amplification method to create a single-step reaction (Joung et al., “Point-of-care testing for COVID-19 using SHERLOCK diagnostics,” 2020). For example, amplification and CRISPR detection may be performed using reagents having compatible chemistries (e.g., reagents that do not interact detrimentally with one another and are sufficiently active to perform amplification and detection). In some embodiments, CRISPR/Cas detection may be combined with LAMP.

### 4. Reagents

According to some embodiments of the present technology, the diagnostic devices described herein may comprise and/or utilize reagents (e.g., lysis reagents, nucleic-acid amplification reagents, CRISPR/Cas detection reagents, and the like) in various test procedures of a diagnostic test. In some embodiments, one or more of the reagents may be contained within a diagnostic device (e.g., in a reaction vial of the diagnostic device). In some embodiments, one or more of the reagents may be provided separately (e.g., in one or more caged caps, in one or more separate vials, etc.). For example, a diagnostic device may be comprised of one or more caged caps comprising one or more lysing reagents and/or one or more amplification reagents.

According to some embodiments of the present technology, at least one (and, in some instances, each) of the reagents used in a diagnostic test may be in liquid form (e.g., in solution). In some embodiments, at least one (and, in some instances, each) of the reagents used in a diagnostic test may be in solid form (e.g., lyophilized, dried, crystallized, air jetted, and the like) and may be activated with buffer fluids prior to or during use.

4.1 Lysing Reagents

According to some embodiments of the present technology, the reagents may be comprised of one or more lysis reagents. A lysis reagent may refer generally to a reagent that promotes cell lysis either alone or in combination with one or more other reagents and/or one or more conditions (e.g., heating). In some embodiments, the lysis reagents may be comprised of one or more enzymes. Non-limiting examples of suitable enzymes may include lysozyme, lysostaphin, zymolase, cellulose, protease, and glycanase. In some embodiments, the lysis reagent(s) may be comprised of one or more detergents. Non-limiting examples of suitable detergents may include sodium dodecyl sulphate (SDS), Tween (e.g., Tween 20, Tween 80), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), Triton X-100, and NP-40. In some embodiments, the lysis reagents may be comprised of an RNase inhibitor (e.g., a murine RNase inhibitor). In some embodiments, a concentration of the RNase inhibitor may be is at least 0.1 U/μL, at least 1.0 U/μL, or at least 2.0 U/μL. In some embodiments, the concentration of the RNase inhibitor may be in a range from 0.1 U/μL to 0.5 U/μL, 0.1 U/μL to 1.5 U/μL, or 1.0 U/μL to 2.0 U/μL. In some embodiments, the lysis reagents may comprise Tween (e.g., Tween 20, Tween 80).

4.2 Contamination-Prevention Reagents

According to some embodiments of the present technology, the reagents may be comprised of at least one reagent that works to reduce or eliminate potential carryover contamination from prior tests (e.g., prior tests conducted with a common apparatus and/or in a same area). In some embodiments, the reagents may be comprised of thermolabile uracil DNA glycosylase (UDG). In some embodiments, UDG may prevent carryover contamination from prior tests by degrading products that have already been amplified (i.e., amplicons) while leaving unamplified samples untouched and ready for amplification. In some embodiments, a concentration of UDG may be at least 0.01 U/μL, at least 0.03 U/μL, or at least 0.05 U/μL. In some embodiments, the concentration of UDG may be in a range from 0.01 U/μL to 0.02 U/μL or 0.01 U/μL to 0.04 U/μL.

4.3 Reverse Transcription Reagents

According to some embodiments of the present technology, the reagents may be comprised of one or more reverse transcription reagents. As noted above, a target pathogen may have RNA as its genetic material, which may need to be reverse transcribed to DNA prior to amplification. In some embodiments, the reverse transcription reagents may facilitate such reverse transcription. In some embodiments, the reverse transcription reagents may be comprised of a reverse transcriptase, a DNA-dependent polymerase, and/or a ribonuclease (RNase). A reverse transcriptase may refer generally to an enzyme that transcribes RNA to complementary DNA (cDNA) by polymerizing deoxyribonucleotide triphosphates (dNTPs). An RNase may refer generally to an enzyme that catalyzes the degradation of RNA. In some embodiments, an RNase may be used to digest RNA from an RNA-DNA hybrid.

4.4 Nucleic-Acid Amplification Reagents

According to some embodiments of the present technology, the reagents may comprise one or more nucleic-acid amplification reagents. In some embodiments, the nucleic-acid amplification reagents may comprise LAMP reagents, RPA reagents, and NEAR reagents, known in the art. In some embodiments, an enzyme (e.g., Bsm DNA polymerase) may serve as an amplification reagent.

4.5 Reagent Stability Enhancers

According to some embodiments of the present technology, the reagents may comprise one or more additives that may enhance reagent stability (e.g., protein stability). Non-limiting examples of suitable additives may include trehalose, polyethylene glycol (PEG), polyvinyl alcohol (PVA), and glycerol.

4.6 Buffers

According to some embodiments of the present technology, the reagents may comprise one or more reaction buffers. Non-limiting examples of suitable buffers may include phosphate-buffered saline (PBS) and Tris. In some embodiments, the buffers may be buffer fluids. In some embodiments, the buffers may have a relatively neutral pH. In some embodiments, the buffers may have a pH in a range from 5.0 to 7.0, 6.0 to 8.0, 7.0 to 9.0, or 8.0 to 9.0. In some embodiments, the buffers may comprise one or more salts. Non-limiting examples of suitable salts may include magnesium acetate tetrahydrate, potassium acetate, and potassium chloride. In some embodiments, the buffers may comprise Tween (e.g., Tween 20, Tween 80). In some embodiments, the buffers may comprise an RNase inhibitor. In some embodiments, Tween and/or an RNase inhibitor may facilitate cell lysis. In a particular, non-limiting embodiment of the present technology, the buffers may comprise 25 mM Tris buffer, 5% (w/v) poly(ethylene glycol) 35,000 kDa, 14 mM magnesium acetate tetrahydrate, 100 mM potassium acetate, and greater than 85% volume nuclease free water.

### 5. Detection Devices

As noted above, according to some embodiments of the present technology, LFA strips (e.g., the LFA strip 180, 954) may be used as assay vehicles to test for whether a target nucleic-acid sequence, corresponding to a pathogen of interest, is present in a sample obtained from a user. In some embodiments, the target nucleic acid-acid sequence may be amplified (i.e., amplicons) prior to detection via an LFA strip. In some embodiments, an LFA strip may provide results that may be read or interpreted in a non-clinical setting by a lay person (e.g., a person not trained in laboratory procedures). LFA strips may be comprised of reagents or substances for indicating the presence (or absence) of a target nucleic-acid sequence. In some embodiments, an LFA strip may be configured to detect two or more different target nucleic-acid sequences.

According to some embodiments of the present technology, an LFA strip useable with the diagnostic devices described herein may be comprised of one or more fluid-transporting layers, which may be comprised of one or more absorbent materials that allow a fluidic sample to move from one end of the LFA strip (e.g., an intake end) to an opposite end of the LFA strip. In some embodiments, fluid movement may be via wicking or capillary action. Non-limiting examples of suitable materials may include polyethersulfone, cellulose, polycarbonate, nitrocellulose, sintered polyethylene, and glass fibers.

According to some embodiments of the present technology, an LFA strip may be comprised of a plurality of sub-regions. In some embodiments, the fluidic sample may be introduced to a first sub-region (e.g., a region in contact with a sample pad) and may subsequently flow through a second sub-region (e.g., a particle conjugate pad) comprised of a plurality of labeled particles. In some embodiments, the particles may be comprised of gold nanoparticles (e.g., colloidal gold nanoparticles). The particles may be labeled with any suitable label. Non-limiting examples of suitable labels include biotin, streptavidin, fluorescein isothiocyanate (FITC), fluorescein amidite (FAM), fluorescein, and digoxigenin (DIG). In some embodiments, as an amplicon-containing fluidic sample flows through the second sub-region, a labeled nanoparticle may bind to a label of an amplicon, thereby forming a particle-amplicon conjugate. In some embodiments, the fluidic sample may subsequently flow through a third sub-region comprised of one or more test lines. In some embodiments, a first test line may be comprised of a capture reagent (e.g., an immobilized antibody) configured to detect a first target nucleic-acid sequence. In some embodiments, a particle-amplicon conjugate may be captured by one or more capture reagents (e.g., immobilized antibodies), and an opaque marking may appear on the first test line. In some embodiments, the LFA strip may comprise one or more additional test lines configured to detect one or more different target nucleic-acid sequences. In some embodiments, the third sub-region of the LFA strip may further comprise one or more control lines. For example, a control line may be a human (or animal) nucleic-acid control line configured to detect a nucleic acid (e.g., RNase P) that is generally present in all humans (or animals). The control line may be used to confirm whether a human (or animal) sample was successfully collected, nucleic-acid sequences from the sample were amplified, and the amplicons were transported through the LFA strip successfully.

According to some embodiments of the present technology, a diagnostic device may be comprised of two or more LFA strips arranged in parallel, such that a sample fluid may flow in each LFA strip independently of the other LFA strip(s).

### 6. Test Kits

According to some embodiments of the present technology, the diagnostic devices described herein may be part of a test kit useable by a lay person, i.e., a person who is not trained in medical and/or laboratory techniques or procedures. The test kit may be a stand-alone test kit that does not require the use of additional laboratory equipment to perform a diagnostic test. In some embodiments, the test kit may be comprised of a swab device (e.g., the swab device 140) and a diagnostic device (e.g., the vial device 100, 102, 103, 104). One or more reagents necessary for the diagnostic test may be provided in the diagnostic device itself (e.g., in a rupturable ampoule 400 held in a cavity of the diagnostic device, in a portion of the diagnostic device confined by rupturable seals, etc.) or may be provided in a reagent carrier (e.g., a caged cap) to be added by a user during a test procedure.

6.1 Heater

According to some embodiments of the present technology, a heater may be provided as part of a diagnostic device. For example, as shown in FIGS. 9 and 10 the heater 960 may be incorporated in the vial devices 103, 104. In some other embodiments, a separate heater may be provided to heat a sample solution (e.g., for lysis and/or amplification). In some embodiments, the heater may be a printed circuit board (PCB) heater. For example, the PCB heater may be comprised of a bonded PCB with a microcontroller, thermistors, and/or resistive heating elements. In some embodiments, the heater may be pre-programmed with one or more heating protocols. For example, the heater may be pre-programmed with a lysis heating protocol and/or an amplification heating protocol. The lysis heating protocol may be a set of one or more temperatures and one or more time periods that facilitate lysis of a sample. The amplification heating protocol may be a set of one or more temperatures and one or more time periods that facilitate amplification of a nucleic-acid sequence. In some embodiments, the heater may be comprised of an auto-start mechanism that performs heating according to a pre-programmed temperature profile needed for lysis and/or amplification upon activation of the auto-start mechanism by a user.

6.2 Instructions & Software

According to some embodiments of the present technology, a test kit may be comprised instructions associated with sample collection and/or operation of a diagnostic device. For example, the instructions may be comprised of directions for handling a swab device to obtain a sample from a subject as well as directions for providing a collected sample to a diagnostic device (or a component thereof) for further processing. The instructions may be provided in any form readable by a user. For example, the instructions may be written or published, verbal, audible (e.g., telephonic), digital, optical, visual (e.g., videotape, DVD, etc.), and/or provided via electronic communications (including Internet or web-based communications). In some embodiments, the instructions may combine graphical information with textual information. In some embodiments, the instructions may be provided as part of a software-based application.

According to some embodiments of the present technology, the instructions may be provided as part of a software-based application that may be downloaded to a smartphone or other type of portable electronic device, and contents of the downloaded application may guide a user through steps to use a diagnostic device and/or to perform test procedures of a diagnostic test. In some embodiments, the instructions may instruct a user when to add certain reagents and how to do so.

According to some embodiments of the present technology, a software-based application may be connected (e.g., via a wired or wireless connection) a diagnostic device to control the diagnostic device or components thereof and/or to read and analyze test results. In some embodiments, the application may be configured to process an image of an LFA strip captured by an imaging device (e.g., a smartphone camera, etc.) and to evaluate the image to provide a positive or negative test result for each of one or more test lines on the LFA strip.

It should be understood that the features and details described above may be used, separately or together in any combination, in any of the embodiments discussed herein.

Some aspects of the present technology may be embodied as one or more methods. Acts performed as part of a method may be ordered in any suitable way. Accordingly, embodiments may be constructed in which acts may be performed in an order different than described or illustrated, which may include performing some acts simultaneously, even though they may be shown or described as sequential acts in illustrative embodiments.

Aspects described in one embodiment may be combined in any manner with aspects described in other embodiments.

Any use of ordinal terms such as “first,” “second,” “third,” etc., in the description and the claims to modify an element does not by itself connote any priority, precedence, or order of one element over another, or the temporal order in which acts of a method are performed, but is or are used merely as labels to distinguish one element or act having a certain name from another element or act having a same name (but for use of the ordinal term) to distinguish the elements or acts.

The indefinite articles “a” and “an,” as used herein in the specification and in the claims, unless clearly indicated to the contrary, should be understood to mean “at least one.”

Any use herein, in the specification and in the claims, of the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Any use herein, in the specification and in the claims, of the phrase “equal” or “the same” in reference to two values (e.g., distances, widths, etc.) should be understood to mean that two values are the same within manufacturing tolerances. Thus, two values being equal, or the same, may mean that the two values are different from one another by ±5%.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. As used herein in the specification and in the claims, the term “or” should be understood to have the same meaning as “and/or” as defined above.

The terms “approximately” and “about” if used herein may be construed to mean within ±20% of a target value in some embodiments, within ±10% of a target value in some embodiments, within ±5% of a target value in some embodiments, and within ±2% of a target value in some embodiments. The terms “approximately” and “about” may equal the target value.

The term “substantially” if used herein may be construed to mean within 95% of a target value in some embodiments, within 98% of a target value in some embodiments, within 99% of a target value in some embodiments, and within 99.5% of a target value in some embodiments. In some embodiments, the term “substantially” may equal 100% of the target value.

What is claimed is:

1. A diagnostic device for performing a rapid diagnostic test, the diagnostic device comprising:
a vial;
a first rupturable container disposed in an internal cavity of the vial and containing a first fluid; and
a test and readout device in fluid communication with the internal cavity of the vial,
wherein the first rupturable container is configured to rupture during a test procedure of the diagnostic test to enable the first fluid to flow into the internal cavity of the vial.

2. The diagnostic device of claim 1, further comprising:
a conduit configured to enable the fluid communication between the internal cavity of the vial and the test and readout device; and
a lateral-flow assay (LFA) strip disposed in the test and readout device, wherein:
an intake end of the LFA strip is located proximate the conduit, and
the test and readout device is comprised of a window through which the LFA strip is visible.

3. The diagnostic device of claim 2, further comprising:
a sample pad located at the conduit and configured to delay movement of the diluted sample fluid to the LFA strip.

4. The diagnostic device of claim 2, further comprising:
a rupturing device disposed in the internal cavity of the vial, wherein:
the first rupturable container is comprised of a rupturable ampoule containing the first fluid, and
the rupturable ampoule is configured to rupture when the rupturing device is pushed against a surface of the rupturable ampoule at a force exceeding a rupture force.

5. The diagnostic device of claim 4, further comprising:
a rupturable sample container configured to fit in the internal cavity of the vial, wherein the internal cavity of the vial is configured such that, when the sample container is fully inserted in the internal cavity of the vial:
the rupturing device ruptures the rupturable ampoule to allow the first fluid to flow into the internal cavity of the vial,
the rupturing device ruptures the rupturable sample container to allow a sample fluid to flow into the internal cavity of the vial,
the first fluid is a diluent fluid, and
the diluent fluid and the sample fluid interact in the internal cavity of the vial to form a diluted sample fluid.

6. The diagnostic device of claim 5, wherein the rupturing device is a sharp object configured to pierce the rupturable ampoule and the rupturable sample container.

7. The diagnostic device of claim 5, further comprising:
a cap configured to seal an opening of the internal cavity of the vial, wherein:
a location of the conduit prevents the diluted sample fluid from reaching the conduit when the vial is in an upright position, and
in an inverted position with the cap sealing the opening of the internal cavity of the vial, the diluted sample fluid is able to reach the conduit.

8. The diagnostic device of claim 2, wherein the first rupturable container is comprised of a first frangible seal confining the first fluid in a first portion of the internal cavity of the vial.

9. The diagnostic device of claim 8, further comprising:
a reagent confined in a second portion of the internal cavity of the vial by a second frangible seal.

10. The diagnostic device of claim 9, further comprising:
a cap configured to seal an opening of the internal cavity of the vial; and
a sample swab extending from an inner surface of the cap and sized to fit in the internal cavity of the vial with the cap sealing the end of the internal cavity of the vial,
wherein the sample swab is configured to rupture the first and second frangible seals to fit in the internal cavity of the vial.

11. The diagnostic device of claim 10, wherein:
the reagent is a lyophilized reagent or a liquid reagent, and
when the sample swab bearing a sample is inserted in the internal cavity of the vial with the cap sealing the opening of the internal cavity of the vial, the first fluid is able to interact with the reagent and the sample to form a sample fluid.

12. The diagnostic device of claim 11, wherein:
a location of the conduit prevents the sample fluid from reaching the conduit when the vial is in an upright position, and
in an inverted position with the cap sealing the opening of the internal cavity of the vial, the diluted sample fluid is able to reach the conduit.

13. The diagnostic device of claim 2, further comprising:
a housing;
a crush effector disposed in the housing and configured to deform the vial to rupture the first rupturable container; and
an actuator disposed in the housing and configured to control movement of the crush effector, wherein:
the vial is disposed in the housing, and
the vial is formed of a deformable material that enables a force to be transferred from the crush effector to the first rupturable container.

14. The diagnostic device of claim 8, further comprising:
a housing;
a first crush effector disposed in the housing and configured to deform the vial to rupture the first frangible seal,
a second crush effector disposed in the housing and configured to deform the vial to rupture the second frangible seal, and
an actuator disposed in the housing and configured to control movement of the first and second crush effectors.

15. A diagnostic device for performing a rapid diagnostic test, the diagnostic device comprising:
a housing;
a deformable vial disposed in the housing;
a first rupturable container disposed in an internal cavity of the vial and confining a first fluid;
a test and readout device in fluid communication with the internal cavity of the vial;
a lateral-flow assay (LFA) strip disposed in the test and readout device; and
a crusher configured to deform the vial to rupture the first rupturable container.

16. The diagnostic device of claim 15, further comprising:
a guide configured to convey a liquid from the vial to the test and readout device; and
a sample pad positioned proximate the guide and configured to delay movement of the liquid to the LFA strip.

17. The diagnostic device of claim 15, further comprising:
a second fluid disposed in the internal cavity of the vial and separated from the first fluid by the first rupturable container.

18. A test kit for performing a rapid diagnostic test, the test kit comprising:
instructions for performing the rapid diagnostic test;
a sample collection device; and
a diagnostic device comprised of:
a vial,
a first rupturable container disposed in an internal cavity of the vial and containing a first fluid, and
a test and readout device in fluid communication with the internal cavity of the vial,
wherein the first rupturable container is configured to rupture during a test procedure of the diagnostic test to enable the first fluid to flow into the internal cavity of the vial.

19. The test kit of claim 18, further comprising:
a rupturable sample container comprised of a second fluid, wherein the sample container is sized to fit in the vial,
wherein the sample collection device is configured to deliver a sample to the second fluid via an opening in the sample container.

20. The test kit of claim 18, further comprising:
a reagent held in a caged cap,
wherein the caged cap is configured to release the reagent to the sample container or to the vial when the caged cap is attached to the sample container or to the vial.
